(19) United States
(12) Patent Application Publication
(10) Pub. No.: US 2010/0028282 A1
(43) Pub. Date: Feb. 4, 2010

(54) HAIR DYEING PROCESS USING A LEAVE-IN ANIONIC COLOURED POLYELECTROLYTE

(76) Inventors: Maxime De Boni, Tokyo (JP); Bruno Laguitton, Kremlin Bicetre (FR); Henri Samain, Bievres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/440,446

(22) PCT Filed: Sep. 17, 2007
(86) PCT No.: PCT/FR2007/051950

§ 371 Date: Aug. 25, 2009

Related U.S. Application Data
(60) Provisional application No. 60/846,754, filed on Sep. 25, 2006.

(30) Foreign Application Priority Data
Sep. 15, 2006 (FR) 0653769

(51) Int. Cl. A61K 8/30; A61Q 5/06

(52) U.S. Cl. 424/70.6

(57) ABSTRACT

The present invention relates to a process for dyeing dark keratin fibres, which comprises the application to the fibres of a composition containing at least one anionic chromophore-containing coloured polyelectrolyte, wherein the application is not followed by a rinsing step. Such a process makes it possible to obtain a coloration which is easy to carry out and which preserves the cosmetic appearance of the keratin fibres.

The invention relates to a process for dyeing hair, using a coloured anionic polyelectrolyte.

The dyeing of keratin fibres and especially of the human hair with dyeing compositions that allow temporary colorations to be obtained is known. The temporary colorations are colorations whose friction resistance or water resistance is low, and are largely removed at the first shampooing. For example, patent applications EP 747 036 and EP 852 843 propose temporary colorations using coloured, water-dispersible polymers which contain sulpho groups and comprise carbonyloxy bonds and a chromophore. U.S. Pat. No. 4,911,731 describes a process for temporary colouring which involves applying to the hair a complex formed from a particular cationic polymer and an anionic coloured polymer, then rinsing the hair thus coloured.

These temporary colorations, also referred to as non-permanent colouring, are not generally used to produce a true coloration: they serve essentially to reinvigorate the fading of a colour which has been obtained by direct or permanent dyeing. They are difficult to see on dark hair.

The aim of the present invention is to provide a new process for the temporary colouring of keratin fibres that does not have the drawbacks of the prior-art colorations—in particular, intense colorations which are visible on dark hair and are resistant to friction or to water, but which are largely removed at the first shampooing and which are easy to employ.

This aim is achieved with the present invention, which provides a process for colouring keratin fibres having a tone level of less than 6, which comprises applying a composition comprising at least one coloured polyelectrolyte having an anionic chromophore, this application not being followed by a step of rinsing of the fibres.

The process of the present invention produces in particular a coloration of dark keratin fibres (tone level less than 6) which is rapid and temporary and which can be employed in a single step without damaging the fibres. The coloration obtained, moreover, is non-marking. In the case of marking on the skin, the coloration is easily removed from the skin by simple rinsing. It is easy to erase with shampooings.

The resulting coloration is visible instantaneously. Drying, for example with a hairdryer or under a hood, can be carried out immediately. The hair can also be dried in the open air. On dark hair, a visible coloration is obtained which is very different from the colouring of the coloured polymer, containing an anionic chromophore, in solution.

The colorations obtained are non-permanent because they start to fade at the first shampooing, and disappear within several shampooings.

The process of the invention in particular produces a coloration which is visible on dark hair which is natural or has been coloured beforehand by, for example, direct dyeing or oxidation dyeing.

By coloured polyelectrolyte containing an anionic chromophore is meant a polymer chain with pendant grafts composed of colouring units, the charge constituting the anionic character of the polymer being carried by the coloured graft.

Where the coloured graft and the polymer chain are charge carriers, the resulting charge must be anionic. There may be a plurality of charges per repeating unit. These polymers may be totally or partially soluble in water. They are generally used in a form in which they have been neutralized by one or more counterions selected from the cations of alkali metals or alkaline earth metals, of hydroxyl-containing or non-hydroxyl-containing organic amines, and ammonium ions.

A coloured anionic polyelectrolyte of this kind comprises, for example, at least one unit represented by:

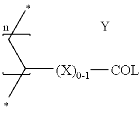

in which COL represents the chromophore, which is attached to the polymer backbone either directly or via a linear or branched, saturated or unsaturated hydro-carbon chain X containing 1 to 10 carbon atoms, preferably 1 to 6 carbon atoms, and in which at least one of the carbon atoms may be replaced by a heteroatom selected from sulphur, silicon, phosphorus, selenium, nitrogen, oxygen, an SO₂ group, the hydrocarbon chain being substituted or unsubstituted, the polymer comprising at least one negative charge carried by the chromophore, and n represents the number of repeating units of this type that are present in the polymer. Generally speaking, n is between 1 and 1000, preferably between 1 and 500. Y is the counterion. These polymers may be in blocks, sequenced or random.

Possible substituents of the carbon chain include alkyl, hydroxyl, alkoxy and hydroxyalkyl radicals, halogens, amino radicals, and amino radicals monosubstituted or disubstituted by an alkyl or hydroxyalkyl radical.

Possible chromophores include the radicals obtained from nitrobenzene, azo, phenothiazine, xanthene, phenanthridine, phthalocyanine dyes, those derived from triarylmethane, and those obtained from direct dyes containing a carbonyl group. The chromophores of this type include, for example, the chromophores obtained from dyes selected from acridone, benzoquinone, anthraquinone, naphthoquinone, benzanthrone, anthranthrone, pyranthrone, pyrazolanthrone, pyrimidinoanthrone, flavanthrone, indanthrone, flavone, (iso)violanthrone, isoindolinone, benzimidazolone, isoquinolinone, anthrapyridone, pyrazoloquinazolone, perinone, quinacridone, quinophthalone, indigoid, thioindigo, naphthalimide, anthrapyrimidine, diketopyrrolopyrrole and coumarin.

In one preferred embodiment the chromophore is substituted by at least one sulphonate, carboxylate, phosphate, phosphonate or sulphate group. Examples include the radicals obtained from acid nitro direct dyes, acid azo dyes, acid azine dyes, acid triarylmethane dyes, acid indoamine dyes, and non-quinone acid natural dyes.

Possible coloured anionic polymers include those described in U.S. Pat. No. 4,911,731, U.S. Pat. No. 6,306,182, EP 852 943, EP 747 036, U.S. Pat. No. 4,381,260, U.S. Pat. No. 4,314,808, U.S. Pat. No. 4,144,252, U.S. Pat. No. 4,051,138.

According to one version the coloured anionic polyelectrolyte corresponds to the formula:

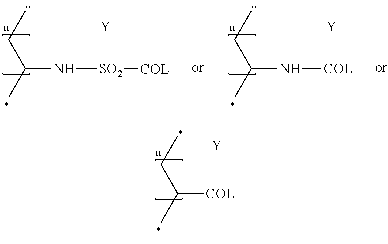

in which COL is an anionic chromophore radical.

Examples of coloured anionic polymers include the following:

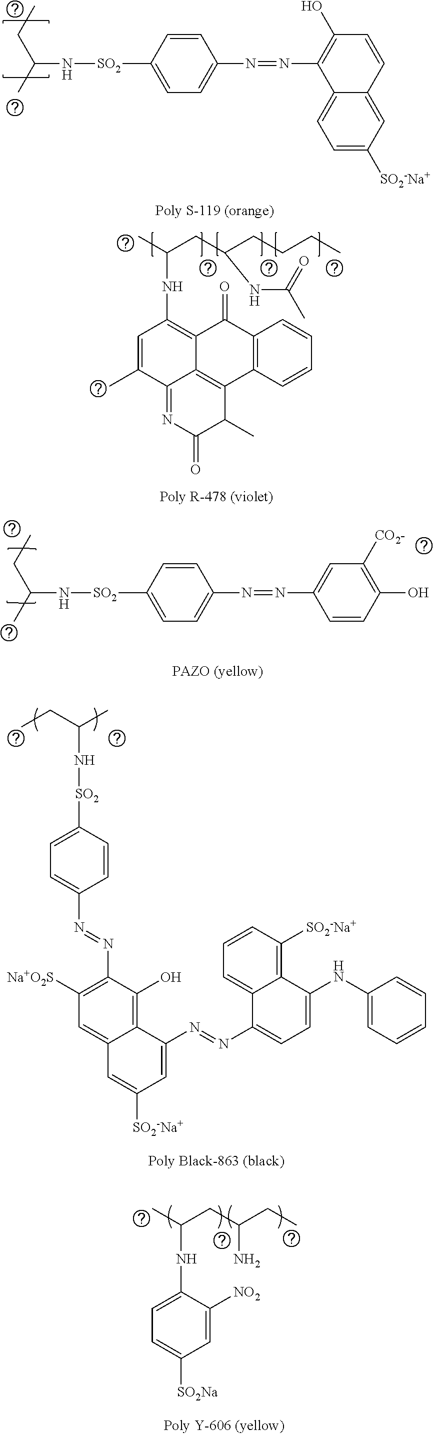

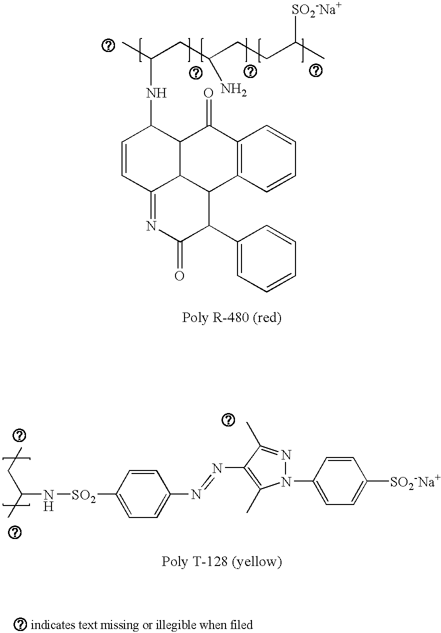

The amount of coloured anionic polymer in the composition which is applied to the fibres is generally between 0.01% to 20%, preferably between 0.1% to 5%. According to one particular embodiment the composition is an aqueous composition.

According to one variant the composition containing the coloured anionic polymer contains one or more organic and/or inorganic salts. As an organic salt, mention may be made of sodium citrate. As an inorganic salt, mention may be made of sodium chloride, ammonium sulphate, magnesium chloride or calcium chloride.

The amount of organic or inorganic salts is generally between 10⁻⁴ and 2 mol/l, preferably between 10⁻³ and 1 mol/l. According to one particularly preferred embodiment the amount of salts is between 10⁻² and 1 mol/l.

The composition useful in the process of the present invention is generally composed of water or of a mixture of water and at least one organic solvent in order to dissolve the compounds that would not be sufficiently soluble in water. Organic solvents include, for example, C₁-C₄ lower alkanols, such as ethanol and isopropanol; polyols and polyol ethers such as 2-butoxyethanol, propylene glycol, propylene glycol monomethyl ether, diethylene glycol monoethyl ether and monomethyl ether; and aromatic alcohols such as benzyl alcohol or phenoxyethanol; and mixtures thereof.

The solvents are, preferably, present in proportions of preferably between 1% and 80% by weight, approximately, relative to the total weight of the composition, and more preferably still between 5% and 60% by weight, approximately.

The compositions which are useful in the process of the invention may also include various adjuvants which are commonly used in compositions for dyeing hair, such as organic or inorganic thickeners, and especially anionic, cationic, nonionic and amphoteric polymeric associative thickeners, antioxidants, penetrants, sequestrants, fragrances, buffers, dispersants, conditioning agents, ceramides, preservatives and opacifiers.

The compositions which are useful in the process of the invention may further comprise at least one thickening polymer, also referred to as “rheology adjustment agents”.

The thickeners may be selected from fatty acid amides (copra diethanolamide or monoethanolamide, ethoxylated alkyl ether carboxylic acid monoethanolamide), cellulosic thickeners (hydroxyethylcellulose, hydroxy-propylcellulose, carboxymethylcellulose), guar gum and its derivatives (hydroxypropylguar), gums of microbial origin (xanthan gum, scleroglucan gum), crosslinked homopolymers of acrylic acid or acrylamidopropane-sulphonic acid, and associative polymers (water-soluble polymers containing hydrophilic zones and fatty-chain hydrophobic zones, which are capable, in an aqueous medium, of associating reversibly with one another or with other molecules).

The compositions which are useful may further comprise at least one surfactant, the surfactants that are suitable for use in the present invention being surfactants that are well known per se (in this respect see, in particular, “Handbook of Surfactants” by M. R. Porter, Blackie & Son (Glasgow and London), 1991, pp. 116-178), their nature not being critical in the context of the present invention.

The compositions which are applied to the keratin fibres may also comprise one or more oxidation dye precursors: one or more oxidation bases and/or one or more couplers. For example, the oxidation bases are selected from para-phenylenediamines, bisphenyl-alkylenediamines, para-aminophenols, ortho-aminophenols, heterocyclic bases and their addition salts.

The oxidation base or bases present are in general present in an amount ranging from 0.001% to 20% by weight, approximately, of the total weight of the dyeing composition, preferably ranging from 0.005% to 6%.

The compositions may comprise one or more couplers which are conventionally used for the dyeing of keratin fibres. Among these couplers particular mention may be made of meta-phenylenediamines, meta-aminophenols, meta-diphenols, naphthalene couplers, heterocyclic couplers and their addition salts.

The coupler or couplers are generally present in an amount ranging from 0.001% to 20% by weight, approximately, of the total weight of the dyeing composition, preferably ranging from 0.005% to 6%.

Generally speaking, the addition salts of oxidation bases and of couplers that can be used in the context of the invention are selected in particular from addition salts with an acid, such as hydrochlorides, hydrobromides, sulphates, citrates, succinates, tartrates, lactates, tosylates, benzenesulphonates, phosphates and acetates, and addition salts with a base, such as sodium hydroxide, potassium hydroxide, ammonia, amines or alkanolamines.

The compositions which are useful may also comprise one or more additional direct dyes, which may in particular be selected from nitro dyes of the benzene series which are neutral, acidic or cationic, direct azo dyes which are neutral, acidic or cationic, direct quinone dyes, and especially neutral, acidic or cationic anthra-quinone dyes, direct azine dyes, direct triarylmethane dyes, direct indoamine dyes and direct natural dyes.

The direct dye or dyes represent in general from 0.001% to 20% by weight, approximately, of the total weight of the aqueous solution in which they are present, more preferably from 0.005% to 10% by weight, approximately.

The pH of the composition which is applied to the fibres is generally between 2 and 12, preferably between 3 and 8. It may be adjusted to the desired value by means of acidifying or alkalifying agents which are commonly used in the dyeing of keratin fibres, or else by means of conventional buffer systems.

The acidifying agents include, for example, organic or inorganic acids such as hydrochloric acid, ortho-phosphoric acid, sulphuric acid, carboxylic acids such as acetic acid, tartaric acid, citric acid and lactic acid, and sulphonic acids.

The alkalifying agents include, for example, ammonia, alkali metal carbonates, alkanolamines such as mono-, di- and triethanolamines, and their derivatives, sodium hydroxide or potassium hydroxide, and the compounds of formula (II) below:

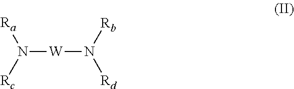

in which W is a propylene residue which is optionally substituted by a hydroxyl group or a C₁-C₄ alkyl radical; Rₐ, R_{b}, R_{c} and R_{d}, which are identical or different, represent a hydrogen atom or a C₁-C₄ alkyl or C₁-C₄ hydroxyalkyl radical.

When the composition comprises at least one oxidation dye precursor or when the intention is to implement a lightening coloration, an oxidizing agent may be employed.

The oxidizing agents which are conventionally used for the oxidation dyeing of keratin fibres are, for example, hydrogen peroxide, urea peroxide, alkali metal bromates, persalts such as perborates and persulphates, peracids and oxidase enzymes, which include peroxidases, 2-electron oxidoreductases, such as uricases, and 4-electron oxygenases, such as laccases. Hydrogen peroxide is particularly preferred.

This oxidizing agent may also be present in one or other of the compositions which are useful in the invention, or may be applied independently.

The oxidizing composition may also contain various adjuvants which are used conventionally in compositions for the dyeing of the hair, and which are as defined above.

According to the process of the invention, the fixing time of each of the compositions is not a limiting factor, since it is possible to obtain the coloration of the keratin fibres instantaneously, allowing the fibres to be dried right after application of the composition comprising the coloured polyelectrolyte. It is, however, possible to allow a fixing time.

According to one particular embodiment the process of the invention may include one or more steps of pre-treatment—for example, a step of treatment with a cationic polymer, it being possible for this or these pre-treatment steps to be followed by a rinsing step.

The process of the invention may be carried out at a temperature of between ambient temperature (20-25° C.) and 200° C., preferably between ambient temperature and 60° C.

In the context of the invention, the process is carried out on dark hair exhibiting a tone level of less than 6, preferably less than or equal to 4. Tone levels are commonly used in hair colouring. They are described in particular in “Science des Traitements Capillaires” by C. Zviak, Masson 1988, p. 278.

This classification is as follows:
1) Black
2) Very dark brown
3) Dark brown
4) Brown
5) Light brown
6) Dark blond
7) Blond
8) Light blond
9) Very light blond
10) Light light blond

The examples which follow serve to illustrate the invention, but without having a limitative character.

### EXAMPLES

### Example 1

An aqueous-alcoholic (50:50) lotion containing 0.5% of poly S-119 is applied to a lock of natural hair containing 90% white hairs. Application is followed immediately by a step of drying with a hairdryer. The orange coloration obtained is very appealing and is removed at the first shampooing.

### Example 2

The lotion of Example 1 is applied to a lock of natural hair containing 90% white hairs, coloured black beforehand (tone level 1). Application is immediately followed by a step of drying (hairdryer). A very bright and very visible green coloration appears instantaneously. This coloration is removed at the first shampooing.

### Example 3

The lotion of Example 3 is applied to a lock of natural hair containing 90% white hairs, coloured black beforehand (tone level 1). Application is immediately followed by a step of drying using a hairdryer. A very bright and very visible midnight blue coloration appears instantaneously. This coloration is removed at the first shampooing.

1. Process for colouring keratin fibres having a tone level of less than 6, which comprises applying a composition comprising at least one coloured polyelectrolyte having an anionic chromophore, this application not being followed by a step of rinsing of the fibres.

2. Process according to claim 1, wherein the chromophore is substituted by at least one sulphonate, carboxylate, phosphate, phosphonate or sulphate group.

3. Process according to either of the preceding claims, wherein the coloured anionic polyelectrolyte is present in the composition in an amount of between 0.01% to 20%, preferably between 0.1% to 5%.

4. Process according to any of the preceding claims, wherein the coloured anionic polyelectrolyte or polyelectrolytes are selected from the following:

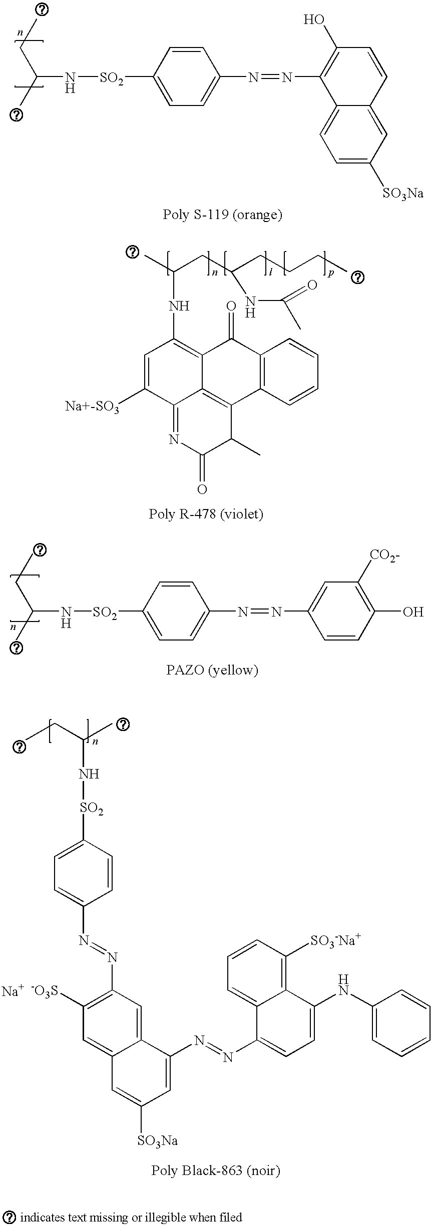

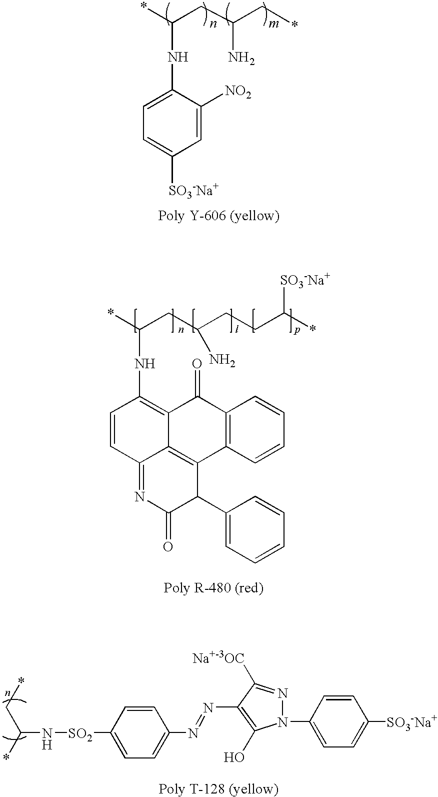

5. Process according to any of the preceding claims, wherein the composition comprises one or more organic and/or inorganic salts.

6. Process according to claim 5, wherein the organic and/or inorganic salts are selected from sodium citrate, sodium chloride, ammonium sulphate, magnesium chloride or calcium chloride.

7. Process according to either of claims 5 and 6, wherein the amount of salts is between 10⁻⁴ and 2 mol/l, preferably between 10⁻³ and 1 mol/l.

8. Process according to any of claims 1 to 7 for colouring hair having a tone level of less than or equal to 4.
